(19) United States
(12) Patent Application Publication
(10) Pub. No.: US 2006/0235423 A1
(43) Pub. Date: Oct. 19, 2006

(54) Apparatus having at least one actuatable planar surface and method using the same for a spinal procedure

(76) Inventor: Alberto Ruiz Cantu, San Francisco, CA (US)

Correspondence Address: COOLEY GODWARD LLP;ATTN: PATENT GROUP, THE BOWEN BUILDING, 875 15TH STREET, N.W. SUITE 800, WASHINGTON, DC, 20005-2221, US

(21) Appl. No.: 11/095,613

(22) Filed: Apr. 1, 2005

(51) Int. Cl. A61B 17/58

(52) U.S. Cl. 606/90

(57) ABSTRACT

A medical device includes a link assembly and an actuating member coupled to the link assembly. The actuating member is configured to actuate the link assembly. A member having a substantially planar surface is coupled to the link assembly. The link assembly is configured to move the member having a substantially planar surface through a range of motion between a first location substantially adjacent the actuating member and a second location at a distance from the actuating member. The substantially planar surface is configured to contact at least a portion of a bone structure (e.g., a vertebra) or a soft tissue area of a patient. A cannula is configured to percutaneously access the patient and the actuating member is moveably disposable within the cannula. The cannula has a cross-sectional shape with at least one substantially planar surface on an inner wall.

### BACKGROUND

The invention relates generally to medical devices and procedures, and more particularly to a minimally invasive medical device that can be used for a variety of medical procedures such as a medical procedure to alter the height of a vertebral body, or a medical procedure to remove material from within a bone structure or soft tissue structure of a patient.

Various known medical devices are configured to repair damaged or collapsed bone structures, such as a collapsed vertebra. To repair a collapsed vertebra, the interior of the vertebra can be expanded to create a cavity within the vertebra. In some cases, bone cement or other material is inserted into the cavity created within the vertebra. Some known devices configured to expand the vertebra, however, require invasive surgical procedures. Invasive surgical procedures are generally less desirable due to the variety of higher risks associated with invasive surgery.

Some known minimally invasive medical devices used for vertebral procedures rely on a point contact, line contact, or an angled surface to contact the vertebra during the expansion process. These types of contact surfaces, however, can cause further damage to the walls of the vertebral body. Thus, a need exists for a controllable and minimally invasive medical device for use in vertebra repair, such as vertebra height restoration. Further, a need exists for a device that provides a sufficient contact surface between the device and the vertebra.

### SUMMARY OF THE INVENTION

A medical device includes a link assembly and an actuating member coupled to the link assembly. The actuating member is configured to actuate the link assembly. A member having a substantially planar surface is coupled to the link assembly. The link assembly is configured to move the member having a substantially planar surface through a range of motion between a first location substantially adjacent the actuating member and a second location at a distance from the actuating member. The substantially planar surface is configured to contact at least a portion of a bone structure (e.g., a vertebra) or a soft tissue area of a patient. A cannula is configured to percutaneously access the patient. The actuating member is moveably disposable within the cannula. The cannula has a cross-sectional shape with at least one substantially planar surface on an inner wall.

### BRIEF DESCRIPTION OF THE DRAWINGS

The present invention is described with reference to the accompanying drawings. In the drawings, like reference numbers indicate identical or functionally similar elements.

FIG. 1 is a schematic illustrating a medical device according to an embodiment of the invention.

FIG. 2 is an exploded view of a medical device according to another embodiment of the invention.

FIG. 3 is an exploded view of a portion of the medical device shown in FIG. 2.

FIG. 4 is a partial exploded view of the medical device shown in FIG. 2.

FIG. 5 is a perspective view of the medical device shown in FIG. 2.

FIG. 6 is a perspective view of the medical device shown in FIG. 2 in a collapsed configuration.

FIG. 7 is a perspective view of the medical device shown in FIG. 2 in a partially expanded configuration.

FIG. 8 is a perspective view of the medical device shown in FIG. 2 in an expanded configuration.

FIG. 9 is a schematic illustrating a medical device according to another embodiment of the invention

FIGS. 10 and 11 are surface views of a medical device according to yet another embodiment of the invention in a collapsed and expanded configuration, respectively.

FIG. 12 is a surface perspective view of a portion of a medical device according to an embodiment of the invention illustrating an actuating device.

FIG. 13 is a cross-sectional view taken along line 13-13 in FIG. 12.

FIG. 14 is a cross-sectional view of an actuating member according to an embodiment of the invention.

FIGS. 15A-15E are cross-sectional views of a cannula according to various embodiments of the invention.

FIG. 16 is a schematic illustrating a medical device according to an embodiment of the invention.

FIGS. 17 and 18 are illustrations of an embodiment of a medical device positioned within a vertebral body to perform a method according to an embodiment of the invention.

FIGS. 19 and 20 are illustrations of an embodiment of a medical device positioned within a vertebral body to perform another method according to an embodiment of the invention.

### DETAILED DESCRIPTION

An apparatus includes a link assembly and an actuating member coupled to the link assembly. The actuating member is configured to actuate the link assembly. The actuating member can be, for example, a rod configured to extend at least partially into a body of a patient and extend partially outside the body of the patient. The actuating member can be hollow (tubular) or solid. The actuating member can be round, square or any other suitable cross section. A member having a substantially planar surface is coupled to the link assembly. The member can be, for example, a mechanical component, such as a plate, coupled to the link assembly. The link assembly is configured to move the member having a substantially planar surface through a range of motion between a first location substantially adjacent the actuating member and a second location at a distance from the actuating member. The substantially planar surface of the member is configured to contact at least a portion of a bone structure or soft tissue structure of a patient.

In some embodiments, the apparatus further includes, a cannula configured to percutaneously access the patient. The actuating member is moveably disposable within the cannula. The cannula has a cross-sectional shape including at least one substantially planar surface on an inner wall. The inner planar surface of the cannula is configured to cooperate with the substantially planar surface of the member.

The apparatus (also referred to herein as a “medical device”) is configured to be used in a variety of medical procedures, such as, for example, a minimally-invasive procedure for vertebral-height-alteration. The medical device also can be configured to maintain disc height while performing a disc or nucleus replacement procedure. The medical device may also be used for procedures in other areas of a patient such as, for example, restoring or altering bone structure or soft tissue, and removing material from within a bone structure or soft tissue area of the patient. The medical device can be used both for procedures performed between bones to distract or displace the bones, or procedures performed within bones. In addition, the medical device may be used to penetrate the internal portion or external area of a bone structure or soft tissue structure. The following description focuses on use of the medical device in spinal procedures, but it should be understood that procedures on other areas of a body may be performed with the medical device.

In one embodiment, the medical device is configured to be inserted into a collapsed or damaged vertebral body and used to spread apart or expand the vertebral body. After the vertebral body has been expanded, bone cement or other suitable material can be filled in the cavity created by the expansion. The medical device includes at least one substantially planar surface configured to contact the vertebral body to provide a large contact surface area. In addition, the cannula included in some embodiments of the medical device includes at least one substantially planar surface on an inner wall that cooperates with or matches the shape of the at least one planar surface. The mating planar surfaces provide for an operatively more robust medical device and improved accuracy during operation of the device. The mating planar surfaces also can provide resistance to torsion or twisting of the medical device within the cannula. In addition or alternatively, the link assembly included in some embodiments of the medical device includes mating gear portions that further improve the robustness of the medical device. The mating gear portions provide a stable and symmetrical displacement of the at least one planar contact surface during operation of the medical device.

The term “link assembly” is used here to mean a set of mechanical components including one or more links. The link assembly can be coupled to other components of the medical device that collectively can be operatively manipulated through a range of motion.

The term “link” is used here to mean a component of the link assembly that is coupled to the actuating member such that the actuating member operatively affects movement of the link. A link can be, for example, a member that is pivotally connected to the actuating member, such that the link translates through a range of motion when the link assembly is actuated. The term “actuating member” is used herein to mean any type of member or structure that actuates the link assembly through its range of motion. Such an actuating member can be, for example, actuatable by a medical practitioner from the proximal end portion of the medical device.

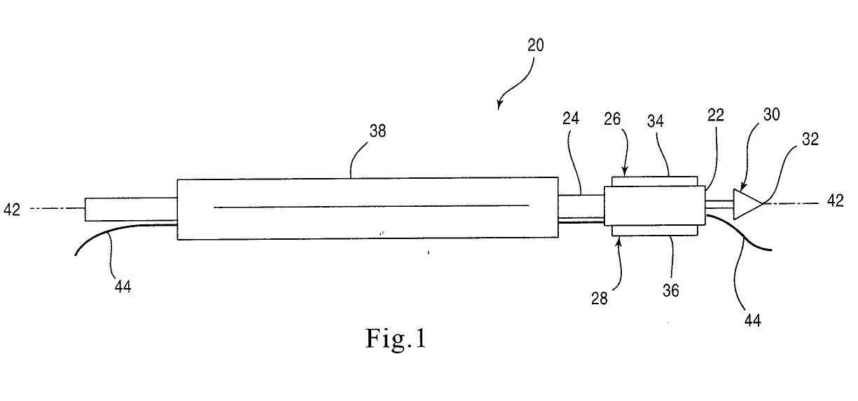

FIG. 1 is a schematic illustration of a medical device 20 according to an embodiment of the invention. Medical device 20 includes a link assembly 22 and an actuating member 24 coupled to the link assembly 22. The actuating member 24 is configured to actuate the link assembly 22. A first member 26 having a substantially planar surface 34 is coupled to the link assembly 22. A second member 28 having a substantially planar surface 36 is coupled to the link assembly 22. First member 26 and second member 28 are coupled to link assembly 22 on opposite sides of a longitudinal axis 42 defined by the actuating member 24. The first member 26 and the second member 28 are configured to move through a range of motion when the link assembly 22 is actuated by the actuating member 24. The substantially planar surface 34 and the substantially planar surface 36 are configured to move from a first location substantially adjacent the actuating member 24 (shown in FIG. 1) to a second location at a distance from the actuating member 24 (not shown in FIG. 1). The substantially planar surface 34 is configured to contact a portion of a vertebra of a patient and the substantially planar surface 36 is configured to contact a portion of the vertebra of the patient at a different location than the portion contacted by the substantially planar surface 34. Although FIG. 1 illustrates both a first and second member 26 and 28 having a substantially planar surface 34 and 36, respectively, it should be understood that the medical device 20 can operate with only one member having a substantially planar surface.

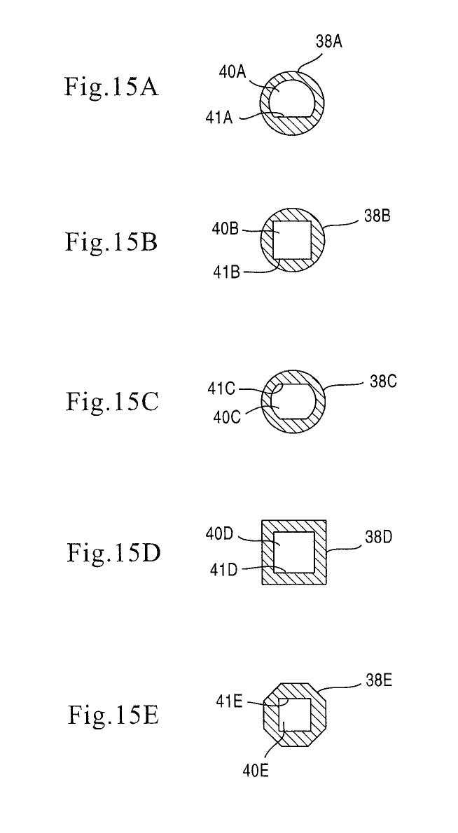

A projection 30 having a sharpened end portion 32 may optionally be coupled to the actuating member 24. The actuating member 24 can be configured to be moveably disposed within a cannula 38, which is configured to percutaneously access a body of a patient in conjunction with a minimally invasive medical procedure. The cannula 38 defines a lumen 40 (examples of which are shown in FIGS. 15A-15E) and a cross-section having one or more planar surfaces 41 on an inner wall of the cannula 38 (examples of which are shown in FIGS. 15A-15E). In some embodiments, the cannula does not include one or more linear surfaces on an inner wall, and thus has a substantially round inner cross-section or diameter. Although a cannula is illustrated in some of the embodiments, a cannula is not necessary to be included in the medical device 20. In an embodiment including a cannula 38, the link assembly 22, the first member 26 and the second member 28 are configured to pass through the lumen 40 of the cannula 38 to a location within the vertebra of the patient. At least a portion of the actuating body 22 is disposed within the cannula 38 during use of the medical device 20. The cannula 38 can be a variety of different configurations. For example, the cannula 38 can have a cross-section having an outer surface that is round, square, octagonal or any other suitable shape, such as those shown in FIGS. 15A through 15E.

Medical device 20 may also include a guidewire 44 coupled to the actuating member 24 and configured to direct the medical device 20 to a selected location on the vertebra of a patient. In such an embodiment, the medical device 20 can be used to percutaneously access the patient's body with the guidewire 44 coupled to the medical device 20. The guidewire 44 may be coupled directly to actuating member 24 by passing through a lumen (not shown in FIG. 1) defined by the actuating member 24. For example, the guidewire 44 can pass through a lumen of the actuating member 24 along the longitudinal axis 42, or alternatively through a lumen positioned along the outer surface of the actuating member 24. Alternatively, the guidewire 44 may be coupled to the link assembly 22, one of the first member 26 and the second member 28, or the cannula 38. In some embodiments, the projection 30 can include a lumen and the guidewire 44 can pass through the lumen of the projection 30. The guidewire 44 may be a typical guidewire used in medical procedures for placement of the medical device in the desired location within the patient's body.

For example, the vertebral body can be percutaneously accessed with the use of a spinal needle assembly. Such a spinal needle assembly can include an outer sleeve and an inner solid core slidably disposed within a lumen of the outer sleeve. After accessing the vertebral body with the spinal needle, the inner core can be removed from the outer sleeve. Guidewire 44 can then be inserted through the lumen of the outer sleeve of the spinal needle and positioned at a desired location within the vertebral body. The medical device 20 can then be coupled to the guidewire 44 as described above and guided to the desired location via the guidewire 44.

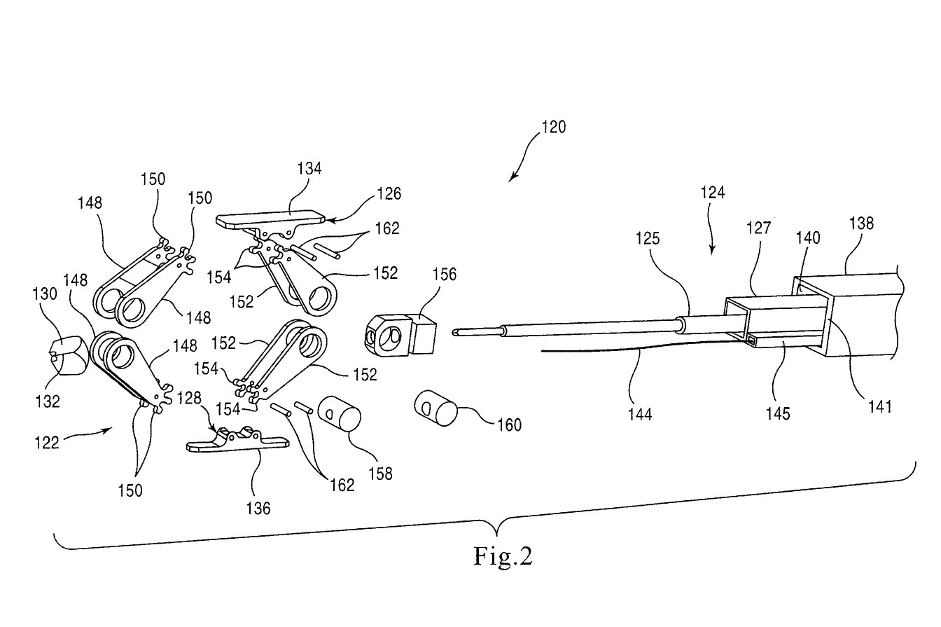

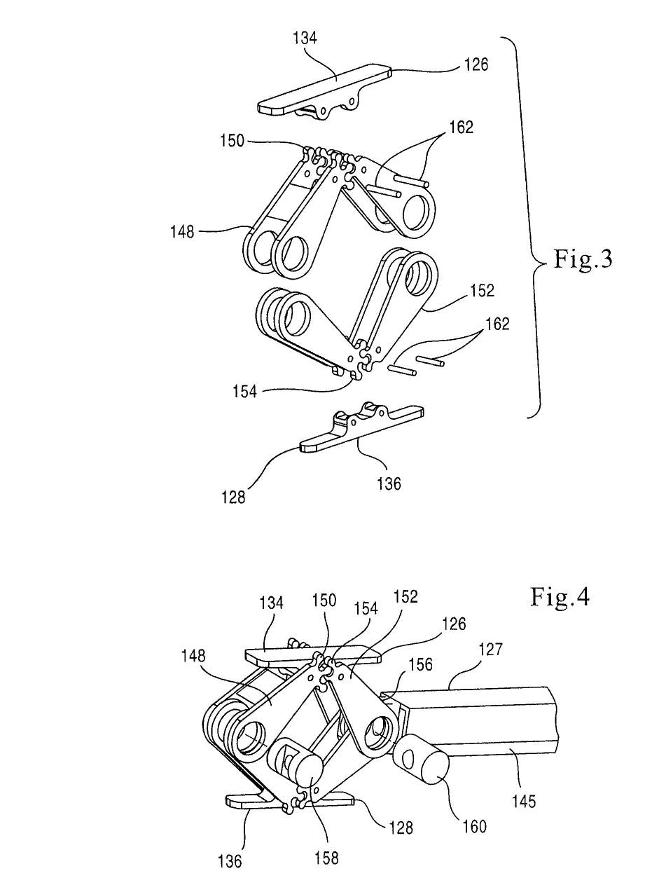

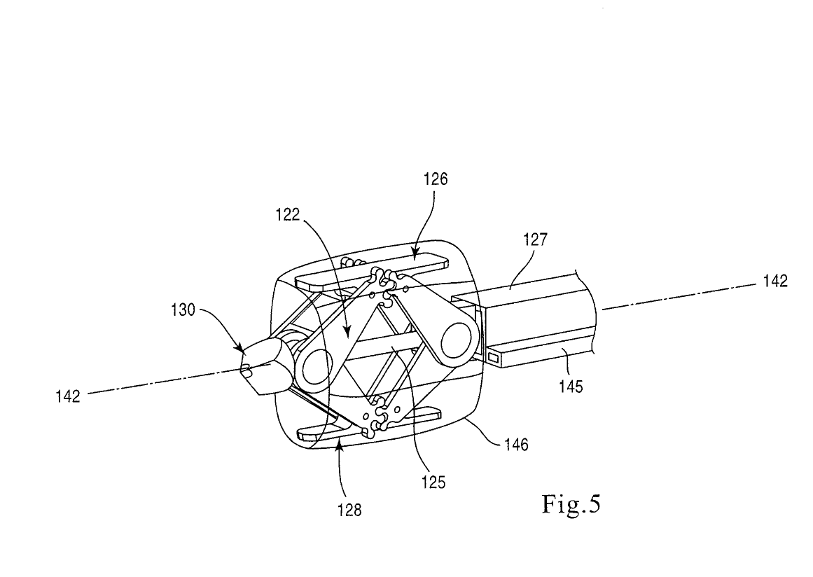

FIGS. 2-4 illustrate various exploded and partially exploded views of a medical device 120, according to an embodiment of the invention. FIG. 5 illustrates an assembled view of the medical device 120 illustrated in FIGS. 2-4 with an optional wrap 146 to be described in more detail below. Medical device 120 includes a link assembly 122, a projection 130 having a sharpened end portion 132, and an actuating member 124 configured to couple to link assembly 122. Medical device 120 also includes a first member 126 having a substantially planar surface 134, a second member 128 having a substantially planar surface 136, and a cannula 138. The cannula 138 is configured to percutaneously access the body of a patient and includes at least one planar surface 141 on an inner wall, as shown in FIG. 2.

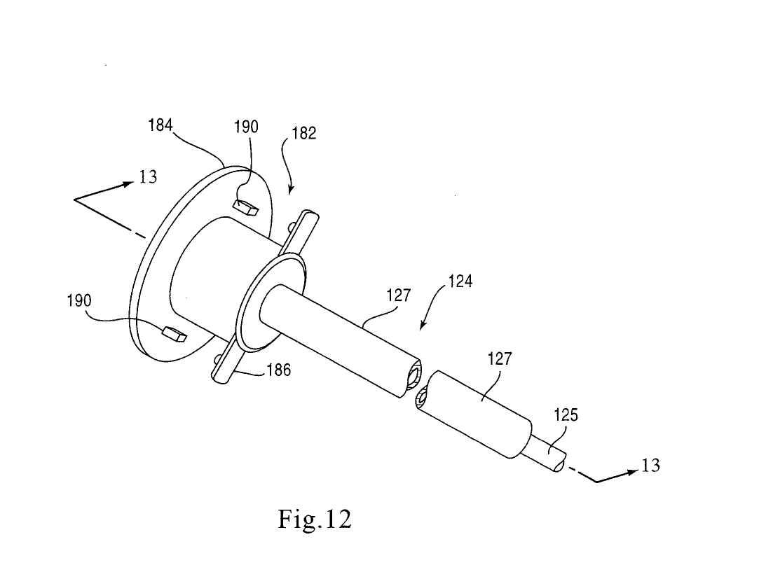

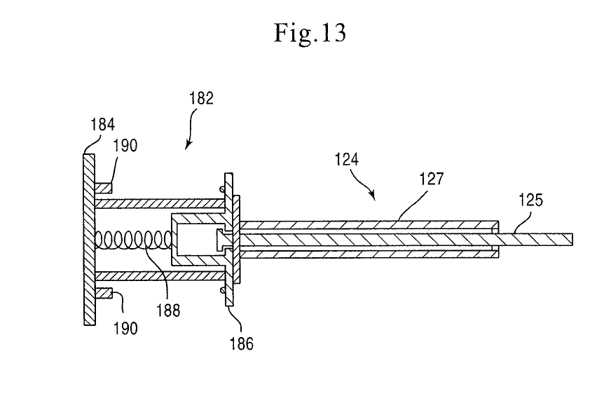

The actuating member 124 includes an outer sleeve 127 and an inner elongate body 125 slidably disposed within a lumen defined by the outer sleeve 127. The actuating member 124 is configured to actuate movement of the link assembly 122. The actuating member 124 can be coupled at its proximal end to an actuating device 182 (shown in FIGS. 12 and 13) that actuates movement of the inner elongate body 125 relative to the outer sleeve 127. The actuating device 182 includes a base 184, an actuating handle 186, and at least one biasing member 188, such as a spring. The biasing member 188 can bias the inner elongate body 125 to a position where it is fully extended from a distal end of the cannula 138. When the inner elongate body 125 is in the fully extended position, the link assembly 122 will be in a closed or collapsed configuration, to be discussed in more detail below. When actuating handle 186 is pulled toward base 184, the inner elongate body 125 will be pulled proximally. When the inner elongate body 125 is pulled proximally, the distal end of the link assembly 122 will be pulled proximally causing the link assembly 122 to be moved to an open or expanded configuration, to be discussed in more detail below. Actuating device 182 also includes a locking element 190 to hold the inner elongate body 125 in the proximal position. Actuating device 182 is one such device that can be used to actuate movement of medical device 120. Other actuating devices configured to initiate movement of a first component disposed within a second component, such as a hydraulic mechanism, may also be used to actuate medical device 120.

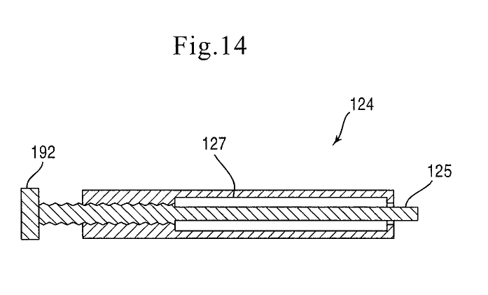

In another embodiment, actuating member 124 may be actuated by threadably coupling the inner elongate body 125 to the outer sleeve 127 as shown in FIG. 14. In this embodiment, a handle 192 can be turned to threadably extend and retract inner elongate body 125 within outer sleeve 127. Alternatively, the inner elongate body 125 may be slidably disposed within outer sleeve 127. The actuating member 124 may then be manually actuated by pulling or pushing the inner elongate body 125 such that it slides distally or proximally within the outer sleeve 127. As with actuating device 182, when the inner elongate body 125 is extended distally from the cannula 138 (by either sliding or threadably extending), the link assembly 122 will be moved to the closed or collapsed configuration. When the inner elongate body 125 is moved proximally (by either sliding or threadably retracting), the distal end of the link assembly 122 is pulled proximally causing movement of the link assembly 122 from the closed or collapsed configuration to the open or expanded configuration.

Returning to FIGS. 2-5, the link assembly 122 includes four links 148 having a gear portion 150. Link 148 is configured to be pivotally coupled to the actuating member 124 at a distal end of the link assembly 122. The link assembly 122 also includes four links 152 having a gear portion 154. Link 152 is configured to be pivotally coupled to the actuating member 124 at a proximal end of the link assembly 122. Link assembly 122 also includes a base mount 156, a distal pivot mount 158 and a proximal pivot mount 160. The base mount 156 is configured to couple the proximal end of the link assembly 122 to the actuating member 124. The distal pivot mount 158 is configured to pivotally couple link(s) 148 on the distal end of the link assembly 122 to the actuating member 124. The proximal pivot mount 160 is configured to pivotally couple link(s) 152 on the proximal end of link assembly 122 to the actuating member 124. Pivot pins 162 are used to pivotally couple the gear portion 150 of the link 148 to the gear portion 154 of the link 152 such that the gear portion 150 and the gear portion 154 matingly engage each other. The pivot pins 162 also couple the first member 126 and the second member 128 to the link assembly 122. The pair of pivot pins 162 coupling each of the first member 126 and the second member 128 to the link assembly 122 maintain the first member 126 and the second member 128 in a stable and substantially parallel relation to each other during operation of the medical device 120.

As assembled, the first member 126 and the second member 128 are positioned on opposite sides of a longitudinal axis 142 defined by the actuating member 124. The substantially planar surface 134 of the first member 126 is configured to contact a portion of a vertebra of a patient, and the substantially planar surface 136 of the second member 128 is configured to contact a portion of the vertebra different from the portion contacted by the substantially planar surface 134 of the first member 126. The substantially planar surfaces 134 and 136 each provide a planar contact surface between the medical device 120 and the vertebra. The planar contact surface area reduces the possibility of damage occurring to the vertebra that often occurs when the surface contact area between the medical device and the vertebra is a line contact, a point contact, an angled surface contact, and the like.

Medical device 120 may also include a guidewire 144 configured to direct the medical device 120 to a selected location of the vertebra of a patient. In this embodiment, the guidewire 144 is coupled to a channel 145 of the outer sleeve 127. The guidewire 144 may alternatively run along the longitudinal axis 142 of actuating member 124 through a lumen defined by the inner elongate body 125 (not shown).

Medical device 120 may optionally include a wrap 146 that collectively encases the link assembly 122, the first member 126 and the second member 128, as shown in FIG. 5. The wrap 146 is shown see-through for illustrative purposes. The wrap 146 is a protective covering that prevents unwanted biological material from entering into the link assembly 122 during use of the medical device 120. The wrap can also be useful for containing the link assembly 122 in the event of a malfunction with the medical device. The wrap 146 may be constructed out of any material suitable for such a medical device and flexible enough to protect the medical device 120 without interfering with its operation. In some embodiments, the wrap 146 can be constructed with a radiopaque material to facilitate remote visualization of the wrap. In some embodiments, the wrap 146 can be open on one or both of the ends of the link assembly 122, or fully enclose the link assembly 122.

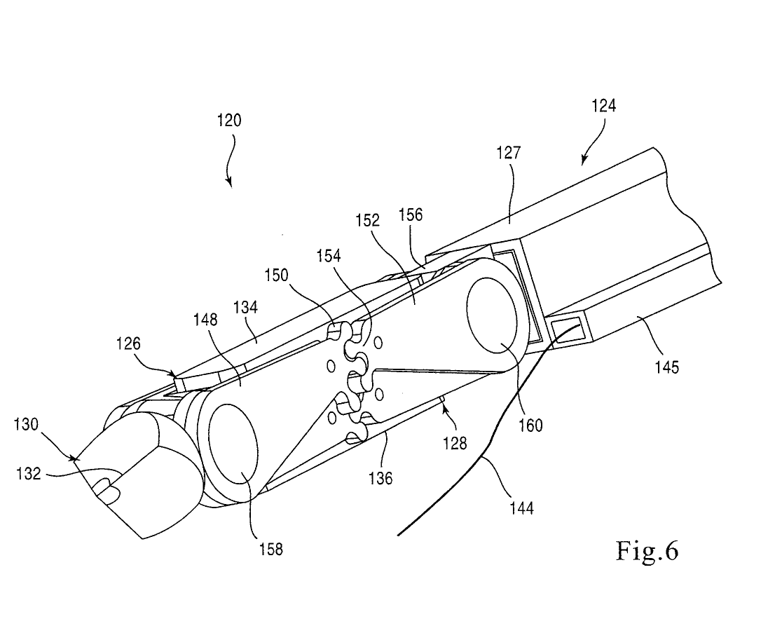

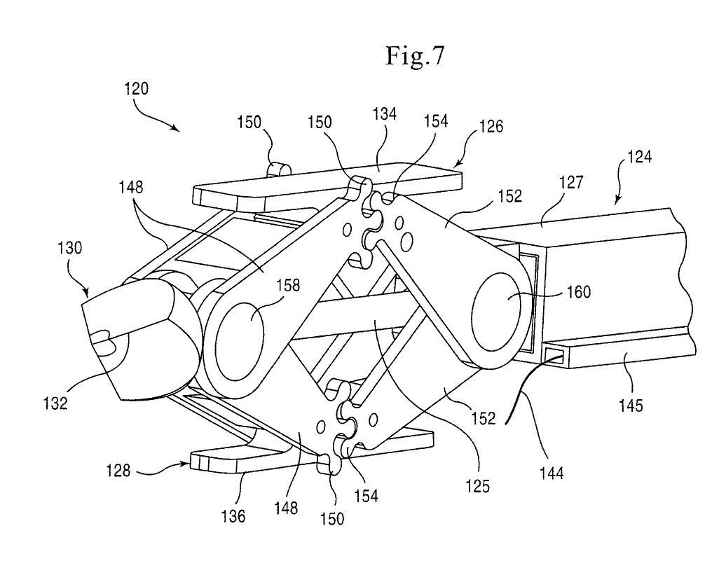

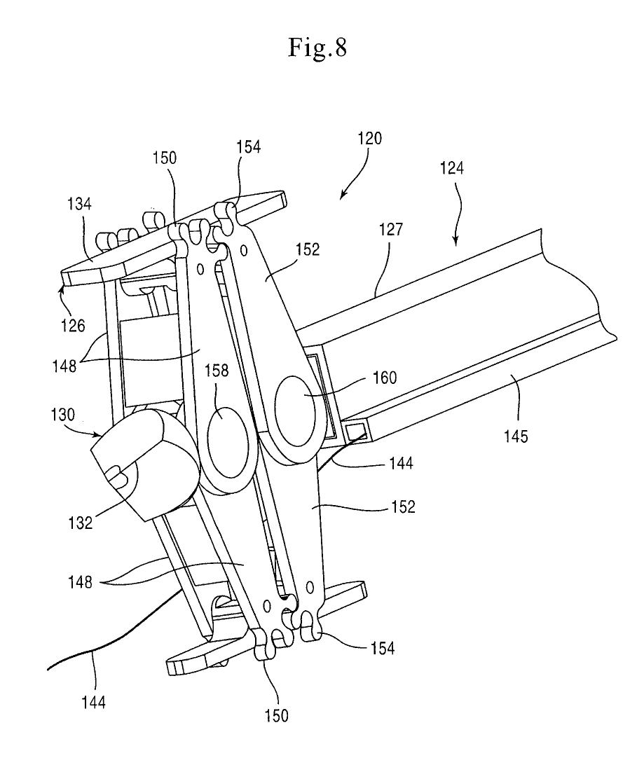

The link assembly 122, the first member 126 and the second member 128 are configured to pass through a lumen 140 of the cannula 138 when the link assembly 122 is in the collapsed configuration. The substantially planar surfaces 134 and 136 of the first member 126 and the second member 128, respectively, are configured to cooperate or match the shape of at least one of the planar surfaces 141 on the inner wall of the cannula 138. For example, an embodiment having two substantially planar surfaces cooperates with a cannula having two interior planar surfaces. The actuating member 124 is at least partially disposed within the lumen 140 while in use, and actuates movement of the link assembly 122 between the collapsed configuration and the expanded configuration. In the collapsed configuration, the first member 126 and the second member 128 are positioned at a first location that is substantially adjacent the actuating member 124 as shown in FIG. 6. In the expanded configuration, the first member 126 and the second member 128 are positioned at a second location at a distance from the actuating member 124 as shown in FIG. 7, or at an alternative second location at a distance from the actuating member 124 as shown in FIG. 8.

FIG. 7 illustrates one possible partially expanded configuration of the link assembly 122 and FIG. 8 illustrates a fully expanded configuration of the link assembly 122. As the link assembly 122 moves between the collapsed configuration and the expanded configuration, the links 148 and 152 pivot about the pivot mounts 158 and 160, respectively, and the gear portions 150 and 154 matingly engage each other to provide for a rotating or swinging motion that is more stable and robust than typical pivoting components. In addition, the mating gear portions 150 and 154 allow for symmetrical displacement of the first member 126 and the second member 128 during actuation of the link assembly 122. Although in this embodiment symmetrical displacement of the first and second members 126 and 128, respectively, is achieved, it should be understood that in some embodiments symmetrical displacement is not necessary for proper functioning of the medical device. For example, in an embodiment having only one such member having a substantially planar surface, no symmetrical displacement would occur.

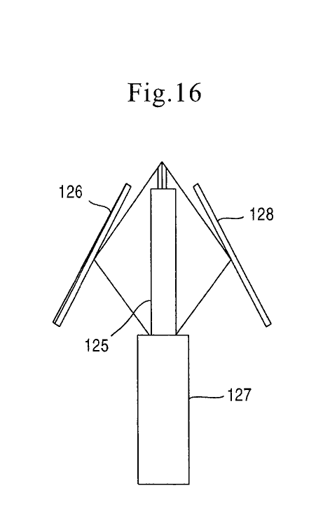

The substantially planar surface 134 of the first member 126 and the substantially planar surface 136 of the second member 128 maintain a substantially symmetrical relationship relative to the actuating member 124 throughout the range of motion of the link assembly 122. In some embodiments, the substantially planar surface 134 of the first member 126 maintains a substantially parallel relation to the substantially planar surface 136 of the second member 128 throughout the range of motion of the link assembly 122 as shown in FIGS. 5-9. In other embodiments, the substantially planar surface 134 of the first member 126 maintains a substantially symmetrical, but not parallel, relation to the substantially planar surface 136 of the second member 128 throughout the range of motion of the link assembly 122, as shown in a schematic illustration in FIG. 16. Further, in some embodiments, during movement of the link assembly 122 through its range of motion, the substantially planar surface 134 of the first member 126 and the substantially planar surface 136 of the second member 128 move a substantially equal distance away from the longitudinal axis 142 defined by the actuating member 124. The substantially planar surfaces 134 and 136 are each configured such that a force exerted on them from contact with the vertebra is transferred to the link assembly 122 substantially uniformly. This can further strengthen the integrity of the medical device 120.

In use, the link assembly 122 in a collapsed configuration, actuating member 124, first member 126 and second member 128 are passed through the cannula 138 to a selected location within a body of a patient, such as a vertebra. If the medical device 120 includes a guidewire (not shown in FIG. 7), the guidewire may be use to direct the link assembly 122 and/or actuating member 124 to the selected location within the vertebra. In addition, if the medical device 120 includes a projection 130 having a sharpened end portion 132, the projection 130 can be used to penetrate the vertebra and provide a path to the desired location.

Once the medical device 120 is positioned in the desired location, the actuating member 124 can actuate movement of the link assembly 122 and the first member 126 and the second member 128. As described above, the link assembly 122 can move between the collapsed configuration and an expanded configuration. Through at least a portion of the range of motion of the link assembly 122, the first and second members 126 and 128, respectively, are moved through a range of motion such that the substantially planar surfaces 134 and 136 move from a location substantially adjacent the actuating member 124 to a location at a distance from the actuating member 124. In the expanded configuration, the first member 126 contacts a portion of the vertebra and the second member 128 contacts a different portion of the vertebra. The substantially planar surfaces 134 and 136 each apply force to the vertebra that distracts the vertebral body and creates a cavity within the vertebra. The force exerted on the vertebra assists in the repair of a collapsed or damaged vertebra by restoring the height of the vertebra. When the repair procedure is complete, the link assembly 122 is moved back to the collapsed configuration so that it can be pulled back through the cannula 138. In addition, the cavity created within the vertebra can be filled with bone cement or other suitable material to further repair the vertebra after the link assembly 122 has been removed from the vertebra. Alternatively, the link assembly 122 can be detached from the medical device 120 and remain with the vertebral body for support.

Medical device 120 may alternatively be used to collect material from within bone structure (e.g., within the vertebra) or soft tissue structure in a patient (e.g., between bones and in a joint or disc). Such material can be, for example, biological material. For this use, the medical device 120 is inserted into a vertebra with the link assembly 122 in a collapsed configuration as described above. The link assembly 122 is then actuated to an expanded configuration and then rotated. The rotation of medical device 120 forces material within the vertebra to be captured within the link assembly 122. The link assembly 122 can also be moved in a for-aft manner (proximally and distally) to scrape and/or loosen material within the vertebra (or within the intervertebral disc when so disposed). The link assembly 122 is then moved to a partially expanded configuration, such that the links 148 and 150 hold the captured material within the link assembly 122. The link assembly 122 is then removed from the vertebra with the captured material. Where a cannula is being used, the link assembly 122 will need to be collapsed sufficiently to enable the link assembly 122 to pass back through the lumen of the cannula for removal.

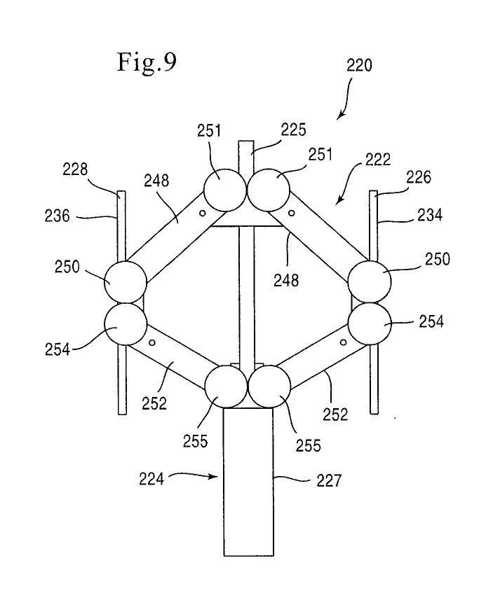

FIG. 9 illustrates a medical device according to another embodiment of the invention. Medical device 220 includes a link assembly 222 and an actuating member 224 coupled to the link assembly 222. Link assembly 222 also includes a first member 226 having a substantially planar surface 234, and a second member 228 having a substantially planar surface 236. Link assembly 222 provides similar functions and includes substantially the same structure as described above for the previous embodiment, except that in this embodiment the link assembly 222 includes two links 248 having a first gear portion 250 and a second gear portion 251, and two links 252 having a first gear portion 254 and a second gear portion 255. The links 248 are pivotally coupled to the actuating member 224 on a distal end of the link assembly 222 as with the previous embodiment, but also include the second gear portion 251 on the distal end of the link assembly 222. The links 252 are pivotally coupled to the actuating member 224 on a proximal end of the link assembly 222 and include the second gear portion 255 on the proximal end of the link assembly 222. The gear portion 250 is configured to matingly engage the gear portion 254, as with the previous embodiment. The gear portion 251 is configured to matingly engage another gear portion 251 on another link 248, and the gear portion 255 is configured to matingly engage another gear portion 255 of another link 252.

In use, the actuating member 224 can be disposed at least partially within a lumen of a cannula (not shown) and actuates movement of the link assembly 222 between a collapsed configuration and an expanded configuration. In the collapsed configuration, the first member 226 and the second member 228 are positioned in a first location that is substantially adjacent the actuating member 224 (not shown). In the expanded configuration, the first member 226 and the second member 228 are positioned in a second location at a distance from the actuating member as shown in FIG. 9. The substantially planar surface 234 of the first member 226 maintains a substantially symmetrical relation to the substantially planar surface 236 throughout the range of motion of the link assembly 222. Further, during movement of the link assembly 222 through its range of motion, the substantially planar surface 234 and the substantially planar surface 236 move a substantially equal distance away from a longitudinal axis defined by the actuating member 224. The substantially planar surfaces 234 and 236 are each configured such that a force exerted on them from contact with the vertebra is transferred to the link assembly 222 substantially uniformly.

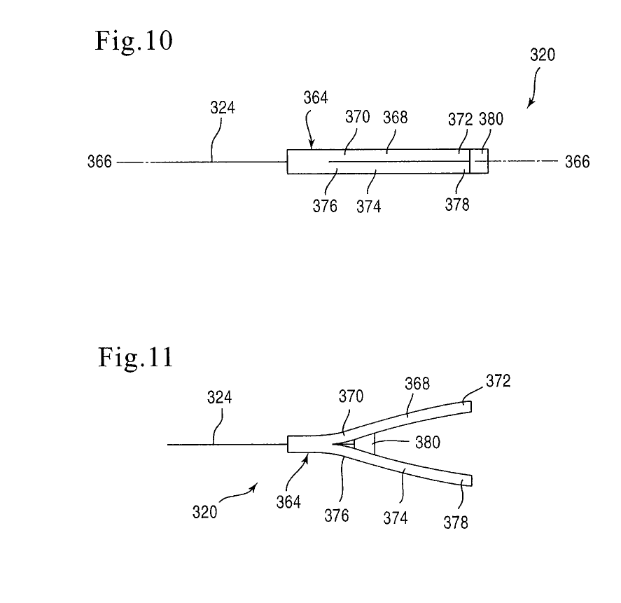

FIGS. 10 and 11 illustrate a medical device according to yet another embodiment of the invention. Medical device 320 includes an actuating member 324 and an arm 364 coupled to the actuating member 324. The arm 364 defines an axis 366 and includes a first portion 368 having a proximal end 370 and a distal end 372, and a second portion 374 having a proximal end 376 and a distal end 378. A mandrel 380 is coupled to a distal end of the actuating member 324. The first portion 368 is configured to contact a portion of a vertebra of a patient and the second portion 374 is configured to contact a different portion of the vertebra of the patient.

The mandrel 380 is configured to move between a first configuration shown in FIG. 10 and a second configuration shown in FIG. 11. In the first configuration, the mandrel 380 is positioned adjacent the distal end 372 of the first portion 368 of the arm 364 and the distal end 378 of the second portion 374 of the arm 364. In the second configuration, the mandrel is moved between the first portion 368 and the second portion 374 of the arm 364 to a position adjacent the proximal end 370 of the first portion 368 and the proximal end 376 of the second portion 374. As the mandrel 380 moves between the first configuration and the second configuration, the first portion 368 and the second portion 374 are forced apart such that the distal end 372 of the first portion 368 and the distal end 378 of the second portion 374 are disposed at a substantially equal distance from the axis 366.

The medical device 320 can include a cannula (not shown in FIGS. 10 and 11) similar to the cannula described above for the previous embodiments. In use, the actuating member 324, the arm 364 and the mandrel 380 are passed through a lumen of the cannula. At least a portion of the actuating member 324 is disposed within the lumen. The arm 364 and mandrel 380 are placed within a vertebra and the actuating member 324 actuates movement of the mandrel 380 to distract, compress, or compact a portion of the vertebra.

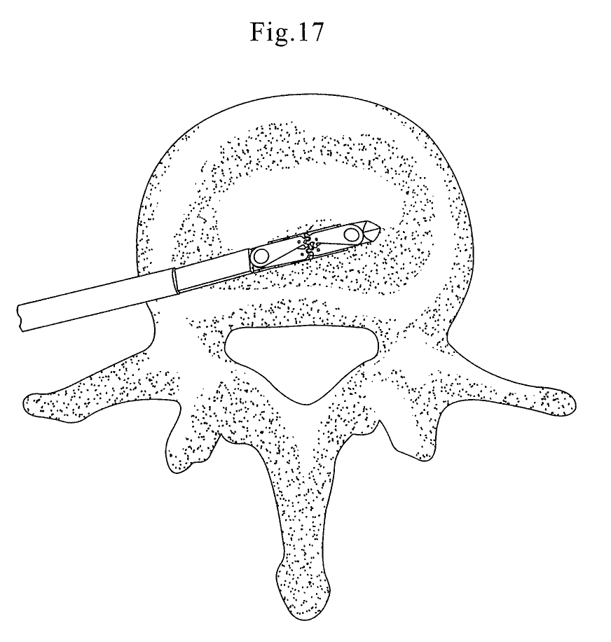

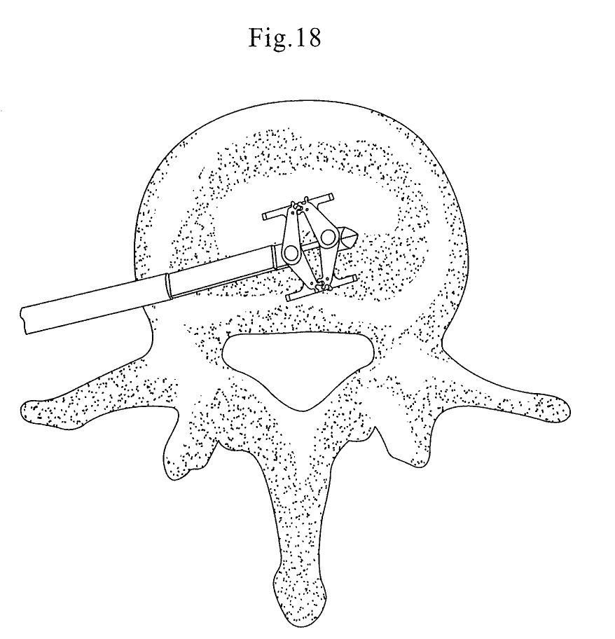

FIG. 17 illustrates a medical device 420 according to an embodiment of the invention inserted outside of the pedicle of the vertebra and into a vertebral body while in a collapsed configuration. FIG. 18 illustrates the medical device 420 after it has been actuated to an expanded configuration. FIGS. 17 and 18 illustrate the medical device as it could be used, for example, in a procedure to create a void in the vertebral body.

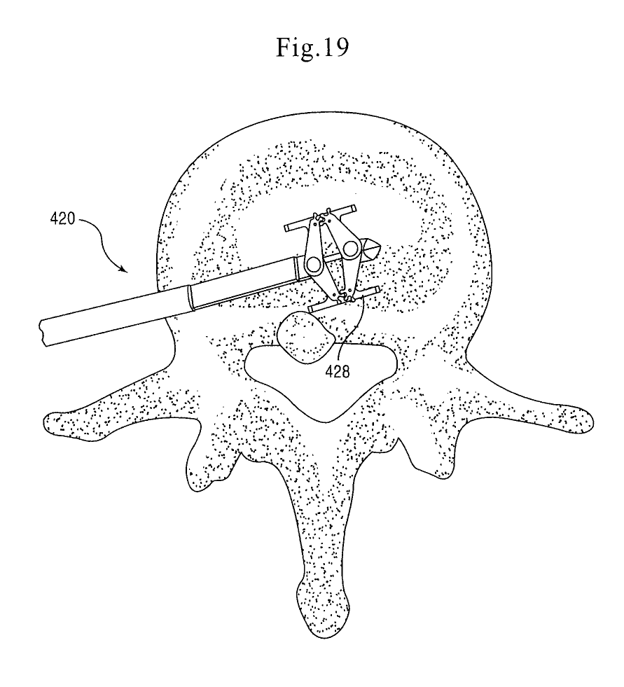

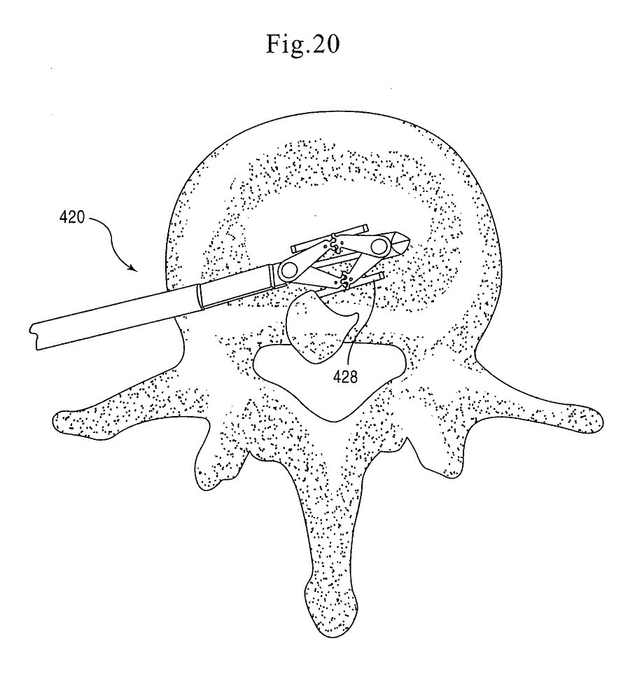

FIGS. 19 and 20 illustrate the medical device 420 as used to retrieve material from within the vertebral body. The medical device 420 is inserted into the vertebral body in a collapsed configuration and then actuated to an expanded configuration such that a member 428 contacts a fragment of material as shown in FIG. 19. The medical device 420 can then be actuated to a collapsed configuration to pull or capture the material within the link assembly, as shown in FIG. 20. The medical device 420 can be fully collapsed (not shown) to remove the medical device from the vertebral body.

The medical device for any of the embodiments may be constructed with any suitable material used for such a medical device. For example, the actuating member (including the inner elongate body and the outer sleeve), the members having a substantially planar surface, the links, the cannula and other components may each be constructed of stainless steel, titanium, ultra high molecular weight (UHMW) plastic, or other suitable biocompatible material. If the medical device is configured to remain within the vertebra, a resorbable material such as calcium phosphate can be used. The projection may be constructed with steel or diamond and can be a variety of configurations such as trocar, diamond, bevel, cone, blunt and the like.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, some embodiments may include only one member having a substantially planar surface configured to contact at least a portion of bone structure (e.g., a vertebra) or soft tissue structure, while other embodiments may have two or more members having a substantially planar surface. In addition, the number of links included in the link assembly may vary and the links may or may not include gear portions. Further, the links may include gear portions on one end or both.

A projection having a sharpened end portion, and a wrap may optionally be included depending on the particular desired use of the medical device. Further, the medical device may include a guidewire to assist with placement of the medical device within a patient, and the guidewire may be coupled to any of the other components of the medical device.

In addition, in some embodiments, a cannula may not be included. In some embodiments, the cannula may have one planar surface on an inner wall, while in other embodiments the cannula may have two or more planar surfaces. For example, the cannula may have an inner lumen having a square cross-section, a triangle cross-section, a semi-circle cross-section and the like. In addition, the cannula can have a variety of different outer surface cross-sectional shapes, such as square, circular or octagonal. The number of planar surfaces on the inner wall of the cannula depends on the number of members having a substantially planar surface that are included on the medical device.

What is claimed is:

1. An apparatus, comprising:
a link assembly;
a first member coupled to the link assembly and configured to actuate the link assembly;
a second member having a substantially planar surface coupled to the link assembly, the link assembly configured to move the second member through a range of motion between a first location at a distance from the first member and a second location at a distance from the first member greater than the distance associated with the first location, the substantially planar surface of the second member configured to contact at least a portion of a vertebra of a patient; and
a cannula configured to percutaneously access the patient, the first member being moveably disposed within the cannula, the cannula having a cross-sectional shape with at least one substantially planar surface on an inner wall.

2. The apparatus of claim 1, further comprising:
a third member having a substantially planar surface, the substantially planar surface of the second member and the substantially planar surface of the third member being disposed substantially symmetrical relative to the first member through the range of motion of the second member.

3. The apparatus of claim 1, further comprising:
a third member having a substantially planar surface coupled to the link assembly, the substantially planar surface of the third member configured to contact at least a portion of the vertebra of the patient.

4. The apparatus of claim 1, further comprising:
a third member having a substantially planar surface, the substantially planar surface of the second member and the substantially planar surface of the third member being configured to move a substantially equal distance away from a longitudinal axis defined by the first member for the range of motion of the second member.

5. The apparatus of claim 1, wherein the second member is configured to transfer a force exerted on the substantially planar surface to the link assembly substantially uniformly.

6. The apparatus of claim 1, further comprising:
a projection having a sharpened end portion coupled to a distal end of the first member, the projection configured to penetrate at least a portion of the vertebra.

7. The apparatus of claim 1, further comprising:
a guide wire coupled to the first member, the guidewire configured to direct the apparatus to a selected location on the at least a portion of the vertebra.

8. The apparatus of claim 1, wherein the link assembly includes a first link and a second link, the first link has a gear portion, the second link has a gear portion, the gear portion of the first link and the gear portion of the second link are matingly coupled.

9. An apparatus, comprising:
a link assembly;
a first member coupled to the link assembly and configured to actuate the link assembly, the link assembly including a first link coupled to the first member, the first link including a gear portion, the link assembly including a second link coupled to the first member, the second link including a gear portion, the gear portion of the first link matingly coupled to the gear portion of the second link, the link assembly having a collapsed configuration and an expanded configuration;
a second member having a substantially planar surface coupled to the link assembly at a first location;
a third member having a substantially planar surface coupled to the link assembly at a second location, the first location and the second location being on opposite sides of the first member; and
the link assembly configured such that the substantially planar surface of the second member and the substantially planar surface of the third member maintain a symmetrical relation relative to the first member during movement of the link assembly between the collapsed configuration and the expanded configuration.

10. The apparatus of claim 9, wherein the substantially planar surface of the second member and the substantially planar surface of the third member each are configured to contact at least a portion of a vertebra.

11. The apparatus of claim 9, wherein the first member defines an axis, the substantially planar surface of the second member and the substantially planar surface of the third member are configured to move an equal distance away from the axis when the link assembly moves between the collapsed configuration and the expanded configuration.

12. The apparatus of claim 9, wherein the substantially planar surface of the second member is configured to contact at least a portion of a vertebra, the substantially planar surface of the third member is configured to contact at least a portion of the vertebra different from the portion of the vertebra associated with the substantially planar surface of the second member, the substantially planar surface of the second member and the substantially planar surface of the third member configured to transfer a respective force exerted on its substantially planar surface to the link assembly substantially uniformly.

13. The apparatus of claim 9, further comprising:
a projection having a sharpened end portion coupled to a distal end of the first member, the projection configured to penetrate at least a portion of a vertebra.

14. The apparatus of claim 9, further comprising:
a wrap, the wrap configured to encase collectively the link assembly, the second member and the third member.

15. The apparatus of claim 9, further comprising:
a cannula configured to percutaneously access a patient, the cannula defining a lumen, the link assembly, the second member and the third member configured to pass through the lumen of the cannula, the cannula having a cross-sectional shape with at least two substantially planar surfaces on an inner wall.

16. An apparatus, comprising:
a link assembly;
a first member defining an axis, the first member coupled to the link assembly and configured to actuate the link assembly, the link assembly including a first link coupled to the first member, the first link including a gear portion, the link assembly including a second link coupled to the first member, the second link including a gear portion, the gear portion of the first link being matingly coupled to the gear portion of the second link, the link assembly having a collapsed configuration and an extended configuration;
a second member having a substantially planar surface coupled to the link assembly at a first location;
a third member having a substantially planar surface coupled to the link assembly at a second location, the first location and the second location being on opposite sides of the axis defined by the first member; and
the link assembly configured such that the substantially planar surface of the second member and the substantially planar surface of the third member move an equal distance away from the axis through a range of motion between the collapsed configuration of the link assembly and the expanded configuration of the link assembly.

17. The apparatus of claim 16, wherein the substantially planar surface of the second member and the substantially planar surface of the third member are each configured to contact at least a portion of a vertebra.

18. The apparatus of claim 16, wherein the substantially planar surface of the second member is configured to contact at least a portion of a vertebra, the substantially planar surface of the third member is configured to contact at least a portion of the vertebra different from the portion of the vertebra associated with the substantially planar surface of the second member, the substantially planar surface of the second member and the substantially planar surface of the third member configured to transfer a respective force exerted on its substantially planar surface to the link assembly substantially uniformly.

19. An apparatus, comprising:
an actuating member;
an arm coupled to the actuating member, the arm defining an axis, the arm having a first portion and a second portion, the first portion of the arm and the second portion of the arm each having a proximal end and a distal end, the first portion of the arm configured to contact at least a portion of a vertebra, the second portion of the arm configured to contact at least a portion of the vertebra, the portion of the vertebra associated with the first portion of the arm being different than the portion of the vertebra associated with the second portion of the arm;
a mandrel coupled to the actuating member, the mandrel configured to move between a first configuration in which the mandrel is positioned adjacent the distal end of the first portion of the arm and the distal end of the second portion of the arm, and a second configuration in which the mandrel is positioned adjacent the proximal end of the first portion of the arm and the proximal end of the second portion of the arm; and
the first portion of the arm and the second portion of the arm being disposed substantially parallel when the mandrel is in the first configuration, the distal end of the first portion and the distal end of the second portion being disposed at a substantially equal distance from the axis when the mandrel is in the second configuration.

20. A method, comprising:
inserting a link assembly into an area of a patient while the link assembly is in a collapsed configuration, the area of the patient being one of a bone structure and a soft tissue area;
moving the link assembly from the collapsed configuration to an expanded configuration;
rotating the link assembly such that material disposed within the area of the patient is captured within the link assembly;
moving the link assembly from the expanded configuration to an intermediate configuration, the material being disposed within the link assembly while the link assembly is in the intermediate configuration; and
removing the link assembly from the area of the patient.
